(19) United States
(12) Patent Application Publication
(10) Pub. No.: US 2024/0242789 A1
(43) Pub. Date: Jul. 18, 2024

(54) ARTIFICIAL INTELLIGENCE MATERIALS ASSISTANT

(71) Applicant: Amatrium Inc., Winchester, MA (US)

(72) Inventors: Nhon Q. VO, Winchester, MA (US); Ha V. BUI, Arlington Heights, IL (US)

(21) Appl. No.: 18/562,080

(22) PCT Filed: May 24, 2022
(86) PCT No.: PCT/US2022/030750

§ 371 Date: Nov. 17, 2023

Related U.S. Application Data
(60) Provisional application No. 63/192,336, filed on May 24, 2021.

(51) Int. Cl. G16C 20/90; G16C 20/70

(52) U.S. Cl. CPC G16C 20/90; G16C 20/70

(57) ABSTRACT

An AI materials assistant comprises a materials database, an optimization engine, an input interface and an output interface. The materials database comprises compositional, manufacturing process, and physical/mechanical properties of a plurality of materials. The optimization engine comprises an Artificial Intelligence (AI) engine in operative communication with the materials database, the AI engine being trained on data in the materials database and their associated compositional, manufacturing process and physical/mechanical properties; and a searching model in operative communication with the AI engine. The user input interface is in operative communication with the searching model for inputting queries regarding potential materials or desired material properties. The user output interface is in operative communication with the searching model for providing materials predicted by the AI engine or material properties predicted by the AI engine to users.

### CROSS-REFERENCE TO RELATED APPLICATIONS

The present application claims the benefit of U.S. Provisional Application No. 63/192,336 filed May 24, 2021, entitled “Artificial Intelligence Materials Assistant”.

### BACKGROUND

Material selection, design and engineering involves the understanding of molecular structures, physical and mechanical properties, and chemical reactions of materials and their respective elements. In general, materials design involves iterative searching aimed at identifying optimal solutions in the design space, which is formed by the material composition, structure, and thermomechanical processing. The goal is to find compositions and structures that achieve the most suitable chemical, physical, and mechanical properties subject to various constraints, including required properties, cost, time, availability, manufacturability, and others.

Much of previous work on materials design and development is through trial-and-error method, in which materials developers use their experience and expertise to come up with a material composition that they think could achieve the target set of properties. Oftentimes, this process is relatively random. That material is then tested to validate its properties. If it doesn't meet the requirements, materials developers must come up with another material composition and test it again. This trial-and-error method could take hundreds of iterations until the right material is identified. Physics-based analytical models and computer simulations have been developed recently to reduce the number of iterations, but materials development continues to be a lengthy, expensive, and challenging process.

Much has been written concerning materials and materials engineering and design. However, designing and developing new materials remains a lengthy and typically expensive process with substantial risk of failure. Thus, there is always a need for tools and methods to guide, assist, and accelerate the design and development of new materials.

### SUMMARY

An AI materials assistant comprises a materials database, an optimization engine, an input interface and an output interface. The materials database comprises compositional, manufacturing process, and physical/mechanical properties of a plurality of materials. The optimization engine comprises an Artificial Intelligence (AI) engine in operative communication with the materials database, the AI engine being trained on data in the materials database and their associated compositional, manufacturing process and physical/mechanical properties; and a searching model in operative communication with the AI engine. The user input interface is in operative communication with the searching model for inputting queries regarding potential materials or desired material properties. The user output interface is in operative communication with the searching model for providing materials predicted by the AI engine or material properties predicted by the AI engine to users.

In some embodiments, the AI engine comprises a Machine Learning algorithm. The Machine Learning algorithm may use one of the following multivariable regressions: linear regression, polynomial regression, logistic regression, quantile regression, Lasso regression, ridge regression, elastic net regression, principal components regression, partial least squares regression, ordinal regression, Poisson regression, negative binomial regression, quasi Poisson regression, Cox regression, Tobit regression, support vector regression, random forest regression, decision tree regression, k-nearest neighbors (KNN) regression, Gaussian process regression. In some embodiments, the Machine Learning algorithm uses Gaussian process multivariable regression. In some embodiments, the AI engine comprises a Machine Learning algorithm using deep learning and/or neural network.

In some embodiments, the AI engine uses a random walk process on a multi-dimensional element/property map. The random walk process may employ a plurality of walkers.

In some embodiments, the predicted material properties include one or both groups consisting of confidence levels and error bars.

In some embodiments, the AI materials assistant further comprises an analysis engine. The analysis engine comprises an analysis AI engine in operative communication with the materials database, the analysis AI engine being trained on data in the materials database and their associated compositional, manufacturing process and physical/mechanical properties; and an analysis model in operative communication with the AI engine; at least one user input interface in operative communication with the analysis model for inputting a plurality of material properties and a user-determined weight for each property; and at least one user output interface in operative communication with the analysis model for providing performance indexes for a plurality materials to users based on materials properties in the materials database and the user-determined weights for the material properties.

### BRIEF DESCRIPTION OF THE DRAWINGS

FIG. 1A is a block diagram of an AI Materials Assistant according to one aspect of the present invention.

FIG. 1B is a block diagram of an AI Materials Assistant according to another aspect of the present invention.

FIG. 1C is a block diagram of an AI Materials Assistant according to another aspect of the present invention.

FIG. 1D is a block diagram of an AI Materials Assistant according to another aspect of the present invention.

FIG. 2 is an illustration of a search user interface according to another aspect of the invention.

FIG. 3 is an illustration of a predict composition user interface according to another aspect of the invention.

FIG. 4 is an illustration of a predict properties user interface according to another aspect of the invention.

FIG. 5 is an illustration of a materials analysis user interface according to another aspect of the invention.

FIG. 6 is an illustration of another view of the materials analysis user interface according to another aspect of the invention.

FIG. 7 is an illustration of a performance index view of the materials analysis user interface according to another aspect of the invention.

FIG. 8 is an illustration of a view of the materials analysis user interface providing a pricing analysis according to another aspect of the invention.

FIG. 9 is an illustration of a view of the materials analysis user interface providing a word cloud according to another aspect of the invention.

FIGS. 10a and 10b illustrate a two dimensional element/property map according to another aspect of the invention.

FIG. 11 illustrates a three dimensional element/property map according to another aspect of the invention.

### DESCRIPTION

An Artificial Intelligence (AI) Materials Assistant comprises a materials database and one or more of a materials optimization engines and/or a materials analysis engine.

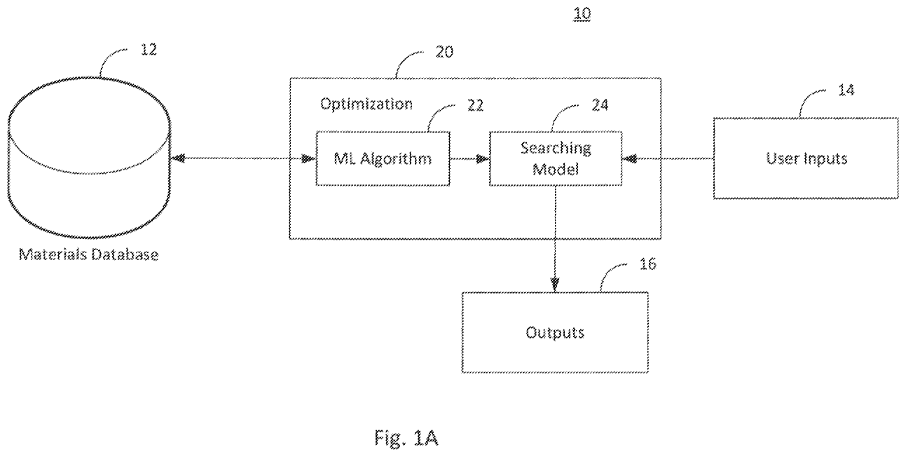

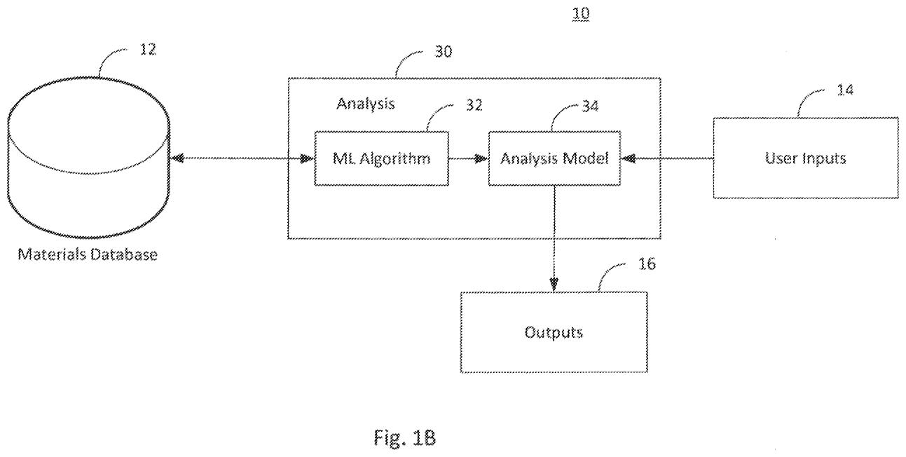

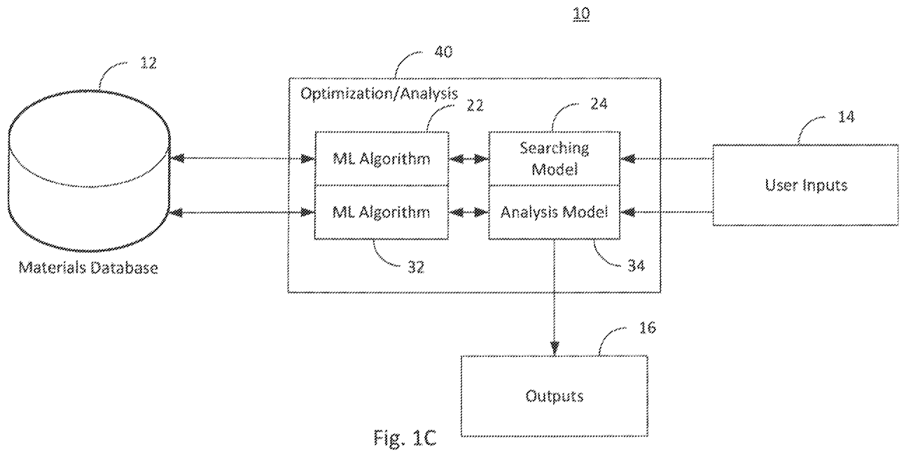

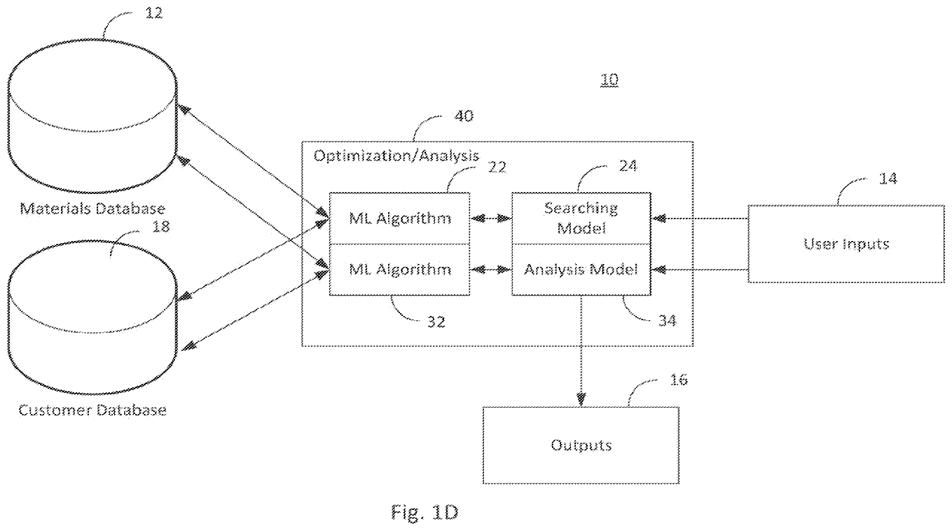

Referring to FIGS. 1A-1C, an AI Materials Assistant 10 comprises a materials database 12, an optimization engine 20, an analysis engine 30, and/or an optimization/analysis engine 40. User input 14 and output 16 interfaces are also provided.

The materials database 12 comprises chemical composition (elements and their proportions) and temper (thermomechanical process and processing steps) of known materials and properties associated with the material. In some embodiments, the materials database 12 includes material names, material categories, the material's physical properties, mechanical properties and chemical composition, available shape, cost related information, and its applications. Physical and mechanical properties include, for example, density, melting point, Poisson ratio, electrical resistivity, electrical conductivity, thermal conductivity, heat capacity, young modulus, yield strength, tensile strength, elongation, shear strength, fatigue strength, machinability etc. Chemical properties include proportions of elements of the material and temper (thermomechanical process and processing steps).

The materials database 12 is populated in a variety of ways, including, for example, by processing existing literature describing materials and their development to extract the chemical composition, temper, and properties of multiple materials. The literature may include handbooks, textbooks, product catalogs, selected journal articles (preferably from trusted sources), research papers, patents and patent publications, web crawling, and other types of published and predicted data from prior knowledge. Information also can be obtained through experimentation and other research techniques. Information also can be obtained through physics-based computational methods such as first-principles calculations, density-functional theory, phase diagrams and thermochemistry, etc.

Referring to FIG. 1A, in some embodiments, the AI Materials Assistant 10 comprises a materials optimization engine 20. The materials optimization engine 20 includes an artificial intelligence (AI) engine 22 and a searching model 24. The AI engine preferably comprises a machine learning model and accesses the materials database 12. In some embodiments, the AI engine 22 is in operative communication with the searching model 24. In some embodiments, the searching model 24 has direct access to the materials database 12. The searching model 24 receives user inputs 14 from one or more users and generates outputs 16.

Referring to FIG. 1B, in some embodiments, the AI Materials Assistant 10 comprises a materials analysis engine 30. The materials analysis engine 30 includes an artificial intelligence AI analysis engine 32 and an analysis model 34. The AI analysis engine preferably comprises a machine learning model and accesses the materials database 12. In some embodiments, the AI analysis engine 32 is in operative communication with the analysis model 34. In some embodiments, the analysis model 34 has direct access to the materials database 12. The analysis model 34 receives user inputs 14 from one or more users and generates outputs 16.

Referring to FIG. 1C, in some embodiments, the AI Materials Assistant 10 comprises a materials optimization/analysis engine 40. The materials optimization/analysis engine 40 combines components of a materials optimization engine 20 and a materials analysis engine 30. Referring to FIG. 1D, in some embodiments, the AI Materials Assistant 10 accesses the materials database 12 and a customer database 18. This provides a customer with the ability to use a proprietary database of chemical composition and temper and properties of materials that it has developed to improve results of the AI Materials Assistant 10 advantageously, this may be done while keeping the customer database 18 confident and without having to add customer proprietary information to the materials database 12. In another example, the AI Materials Assistant 10 accesses the customer database 18, without accessing materials database 12.

In a material search use case, the searching model may accept a set of desired properties and return a list of existing candidate materials, including material names and properties. The materials may be ranked according to closeness to values being searched. User inputs comprise one or more of material names, desired physical properties, mechanical properties and/or chemical composition. Inputs also comprise desired shape or cost. In some embodiments, properties are selected and values entered into one or more web forms.

In some embodiments, the searching model is linked to one or more online stores. For example, a description of a material located by the searching model may include a link to a site where that material may be purchased. In some embodiments, the searching model is embedded into one or more online stores, in form of a chat box, chatbot or similar. For example, a description of a material in the chatbot located by the searching model may include a link to that material on the online store, which can be purchased from that online store.

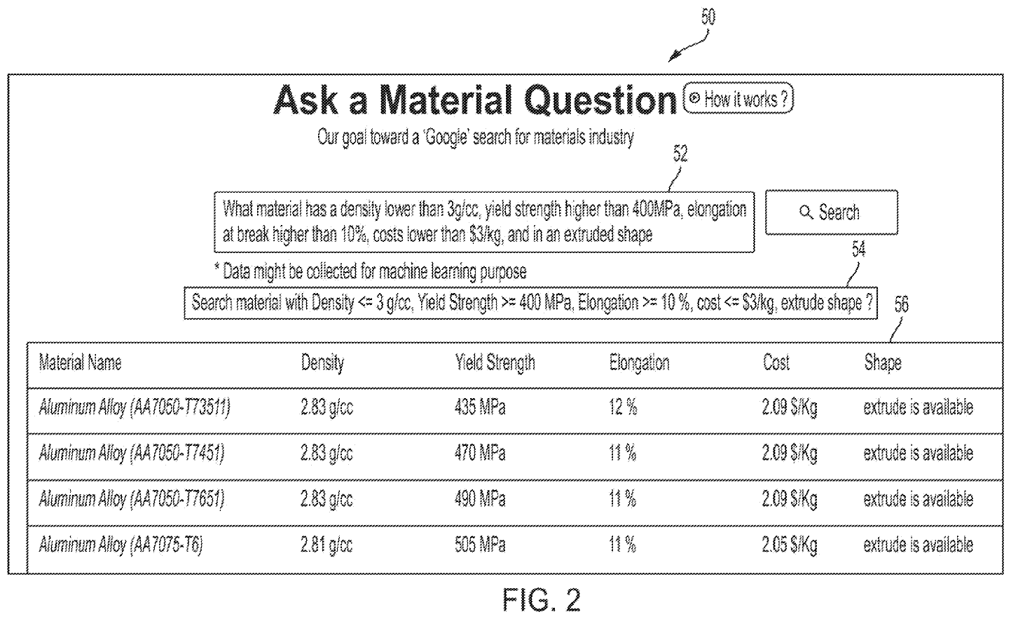

In some embodiments as illustrated in FIG. 2, the search model 24 includes a materials search user interface 50. Desired properties are entered into a search box 52 for a natural language processor, which parses the search segment and formulates a search string illustrated in box 54. For example, a user may type in: “What material has a thermal conductivity higher than 100 w/m-k and a tensile strength greater than 400 mpa?”. The natural language processor processes this language into a search string, as illustrated in FIG. 2, the search string may be displayed to the user in box 54 for confirmation. In the above example, the search string may comprise “Thermal Conductivity >=100 W/m-K, Tensile Strength >=400 MPa”. Materials responsive to the search are displayed in results view 56, including a material name and the properties entered into the search.

In some embodiments, a multidimensional property boundary, such as a Pareto Front, may be generated and displayed. A user may enter two material properties having an effect on each other, for example, conductivity and tensile strength. Values of conductivity may be displayed on one axis and values of conductivity may be displayed on an orthogonal axis increasing one property may have the effect of causing a decrease in the other property. A boundary of maximum (or minimum) values may then be identified and displayed.

The natural language processor may be embedded in the search model 24 or the AI engine 22. In some embodiments, a chat box, or chatbot, may be provided to receive natural language queries and/or revise search terms. The chat box may use programmed responses to user inquiries. Additionally, the chat box may forward questions to a person, such as a subject matter expert to answer the questions from the user.

In some embodiments, the natural language processor is trained, or learns, with a new expression entered by a user, for which it hadn't been trained yet. Then when the new expression is entered by another user in the future, the natural language processor will understand and accurately parses the search segment and formulates a search string. In some embodiments, training the natural language processor with new search expressions is programmed to be automatic (i.e., self-training or self-learning).

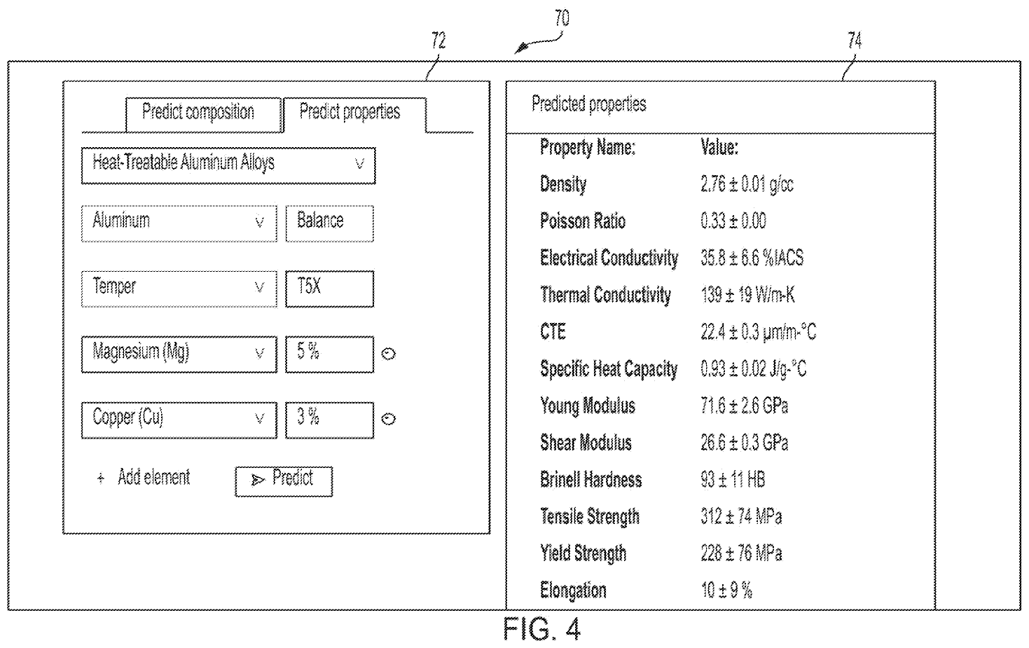

In some embodiments as illustrated in FIG. 4, the AI engine 22 is trained with a Predict Properties User Interface 70 and algorithm to predict the properties of proposed materials. A user proposes materials by entering in interface 72 proposed material elements (e.g., aluminum, copper) and temper (e.g., T5X). Predicted properties are provided in output view 74. To achieve accurate properties predictions, the AI engine 22 is trained on measured properties of existing materials having a known composition and temper. In some embodiments, multivariable regression is applied to each property from each instance of chemical composition and temper in the database. Having been so trained, the AI engine 22 can predict properties for materials that have a chemical composition and temper that are different from the known materials in the database.

In some embodiments, the multivariable regression that is applied in AI engine 22 is linear regression, polynomial regression, logistic regression, quantile regression, Lasso regression, ridge regression, elastic net regression, principal components regression, partial least squares regression, ordinal regression, Poisson regression, negative binomial regression, quasi Poisson regression, Cox regression, Tobit regression, support vector regression, random forest regression, decision tree regression, k-nearest neighbors (KNN) regression. In other embodiments, Gaussian process regression is applied to train the AI engine 22. The benefit of Gaussian process regression is its ability to calculate uncertainty of the property's predictions, which is highly useful for users. Parameters within the Gaussian process can be tuned to achieve the highest accuracy for the property's predictions for each set of the database. In other embodiments, other machine learning models such as deep learning and/or neural network is applied to train the AI engine 22.

Predicted properties may be provided with an error range or confidence interval. In some embodiments, when predicted properties have a large error range or poor confidence interval, the predicted materials are flagged as requiring experimentation and physical testing for validation. In some embodiments, predicted materials having unusual or unexpected beneficial properties are flagged for prioritized experimentation and validation. In some embodiments, after manufacturing a predicted material as a physical material, results of testing and determining actual properties of the material are entered into the materials database, along with the actual components and processes used to make the material. Optionally, this validation information is associated with the prediction to provide learning feedback to the AI engine 22.

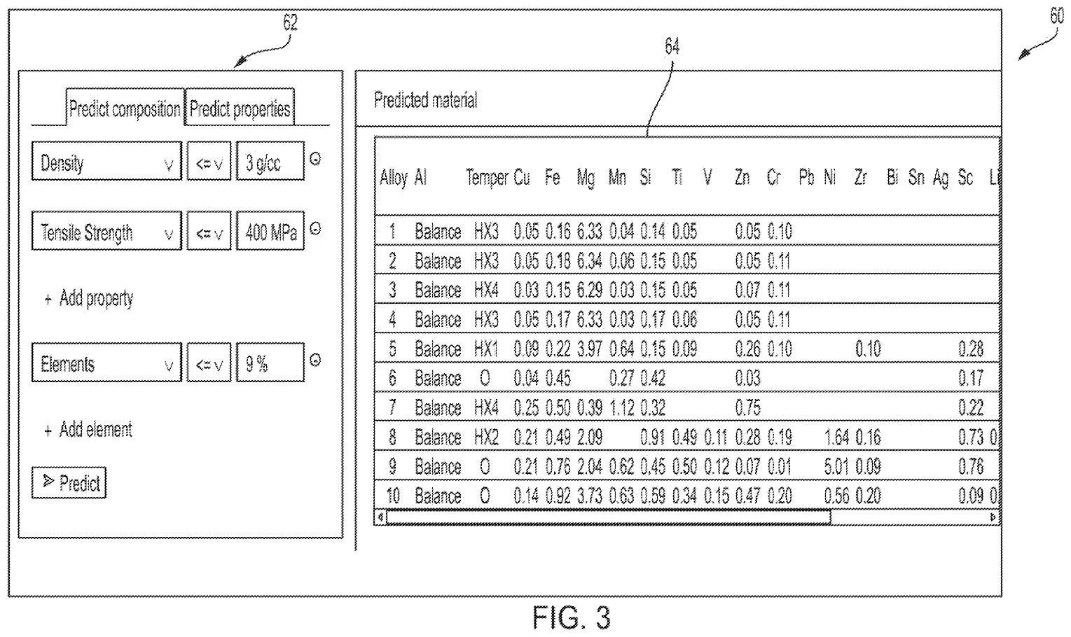

In some embodiments, the materials optimization engine is configured with a Predict Composition algorithm to predict chemical composition and temper of a material corresponding to a set of desired properties. In this case, referring to FIG. 3, a user enters into a user interface 60 desired physical and mechanical properties, and a range of composition and thermomechanical properties. The AI engine generates a list of candidate materials 64, including material composition and thermomechanical process.

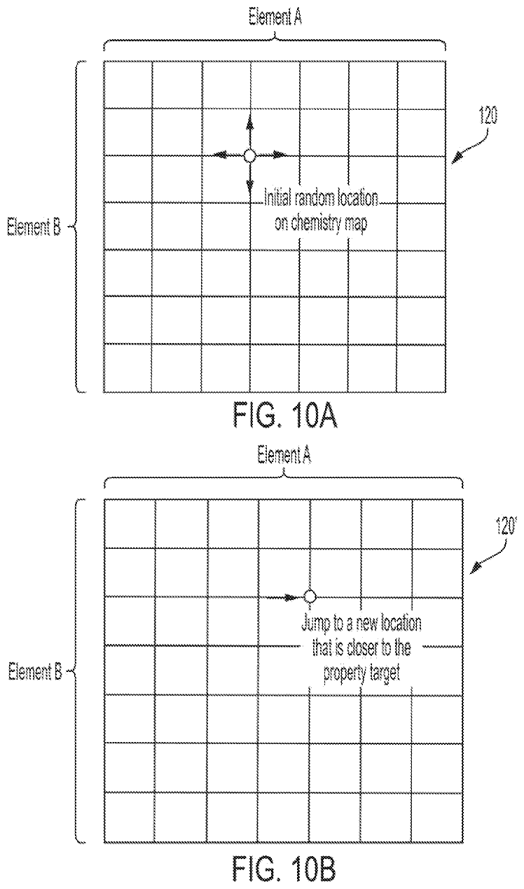

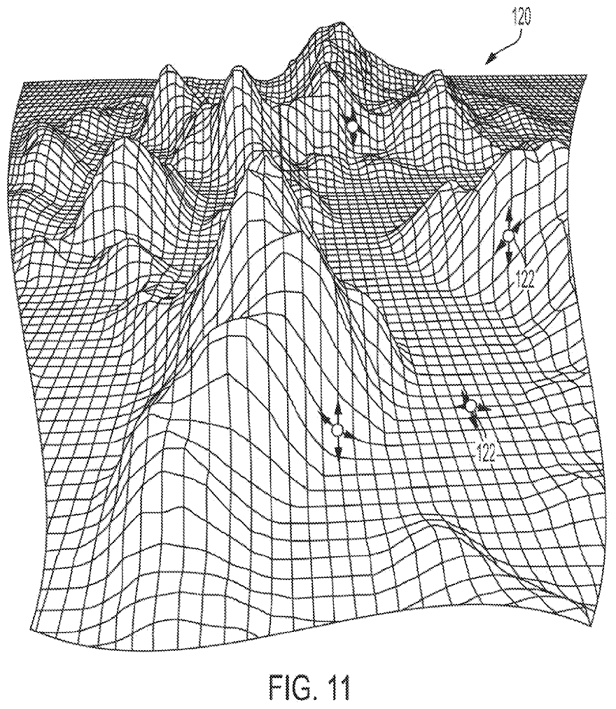

In some embodiments, the materials optimization engine is configured to perform a random walk process on a multi-dimensional element/property map. Referring to FIGS. 10a, 10b, and 11, the element/property map, X and Y coordinates correspond to proportions of individual elements in a material, and the Z coordinate corresponds to a property. In FIG. 10a, for example, a proportion of Element A is represented on the X axis, and a proportion of Element B on the Y axis of property element map 120. A property dependent on the relative proportions would be represented on an axis orthogonal to the X and Y axes. See FIG. 11.

In the random walk process, a “Walker” 122 is placed at initial position, chosen at random on the map. The position represents a composition. The predicted properties of the location/composition are generated. The “Walker” 122 then moves to nearest neighbors in the X-Y lateral dimension (element proportion) randomly (FIG. 10b) and the Z vertical dimension (property value) is again evaluated. Locations (X, Y values) with predicted property values closest to desired properties are identified. Once peak Z values are identified, the X-Y values corresponding to the peak Z values are the predicted composition.

The element/property map may have several localized peaks. Referring to FIG. 11, multiple Walkers 122 may be used to find all of the peaks (and avoid getting stuck on a localized peak). The highest peak from all the Walkers 122 is the best predicted chemical content.

While a simplified element/property map is used to illustrate the invention, in practice more dimensions are required. In some embodiments, the map includes a lateral dimension for each element/temper in the materials database and a vertical dimension for each property in the materials database. The Walker then walks in multiple dimensions. In one example, 18 elements are included in the element/property map and the Walker walks in 18 dimensions. The element/property map also includes all properties under consideration for the material design, as all properties are interconnected.

In this example, the Walker is placed randomly in the 18-dimension space. This assigns the Walker a composition with proportions of 18 constituent elements (some of which may be zero). The properties for that assigned composition are then predicted by the AI engine using the Predict Properties algorithm as set forth above. Then the Walker will walk one step plus or minus for each of the 18 elements around its current location. All properties of each new potential location (i.e., composition) are predicted, again using the Predict Properties algorithm. Then the best composition of this set of predicted properties is chosen based on closest distance between the set of properties of that composition to the target properties, using a distance function. The Walker is then assigned to the location of best composition. By repeating the same procedure, the Walker will keep walking on the 18-dimension space until it finds a location (composition), when the distance between the set of properties of that composition to the target properties can't be any closer, that is, it has reached a “peak”.

In some embodiments, the algorithm is configured to use a longest distance mode in certain circumstances. In one example, if the Walker 122 reaches a location/composition where the new set of properties are all predicted to be better than the target properties, then the Walker will find the longest distance instead of shortest distance, because the longest distance will give an even better set of properties in that case since all properties already exceed the target properties.

A fitting model is used for each group of materials. Different groups of materials may have different fitting equations. The AI engine 22 is configured to understand the material and select appropriate parameters. Referring to FIG. 4, in use, the user inputs a composition for a proposed material. In some embodiments, thermomechanical processes are (e.g., temper) also entered. The AI engine 22 accesses the materials database and predicts the physical properties of the proposed materials and mechanical properties of the proposed material. Inputs may be varied until desired predicted properties are achieved. The use may then fabricate the proposed material and measure the physical material's properties.

In some embodiments, access to subject matter experts is provided. In one example, access is provided on a subscription basis and a set number of questions per month (e.g. five) would be considered within the subscription and answered without further cost. In some embodiments, submitted questions may be posted to a question distribution service and claimed by a subject matter expert. In some embodiments, the AI engine is trained on previous questions and answers and assists in drafting questions to specific experts. In some embodiments, the AI engine is trained on matching the question to the specific expert with a high probability that the specific expert could be able to answer the question accurately. The matching model is based on the content of the question and the knowledge and experience background of the expert. The matching model helps the question distribution service to automatically distribute a large number of questions (e.g. >5,000 question per month) to a large network of experts (e.g. >100 experts). Subject matter experts may be compensated on a per-question basis. Users may rate the subject matter experts. Poorly rated experts may be denied access to question distribution service.

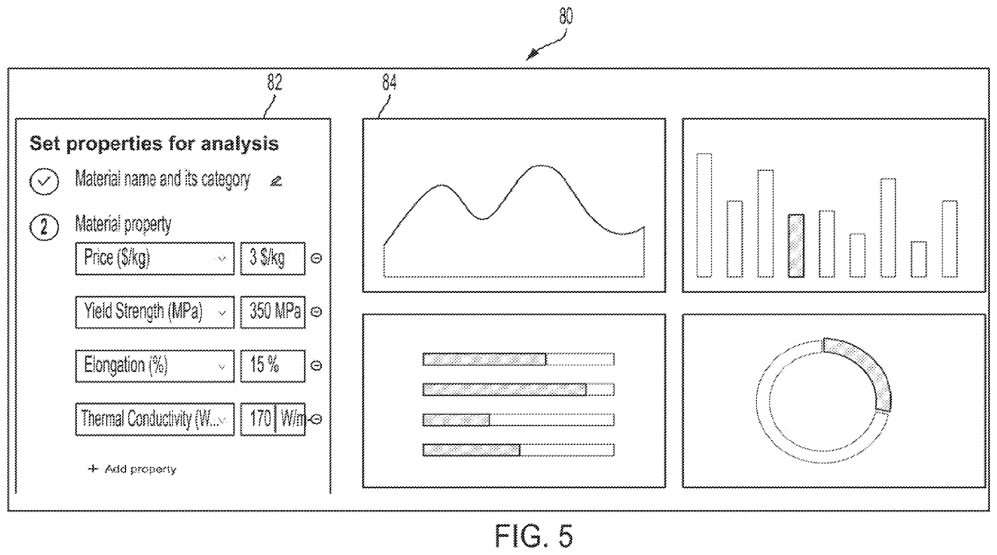

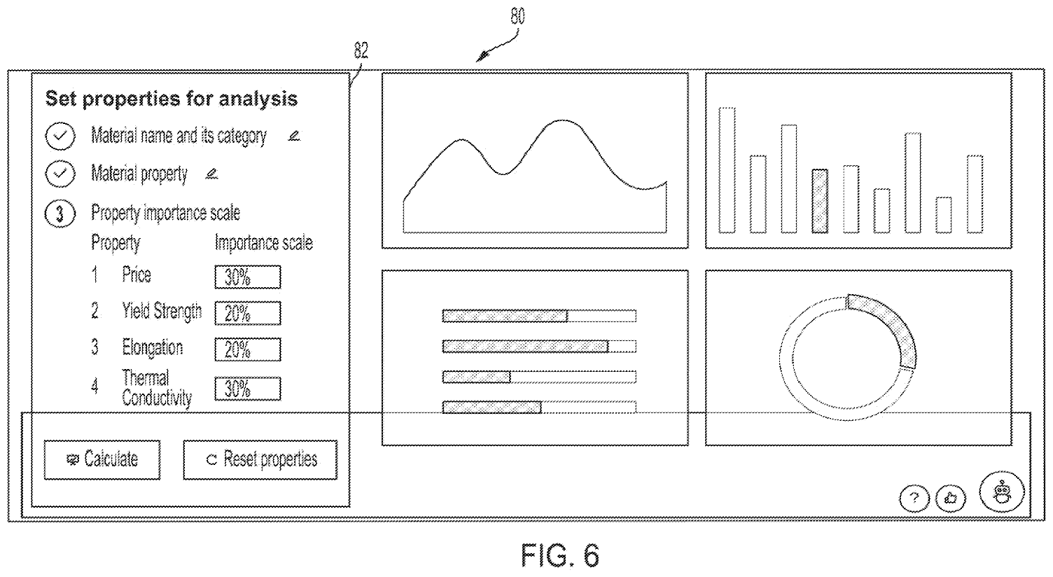

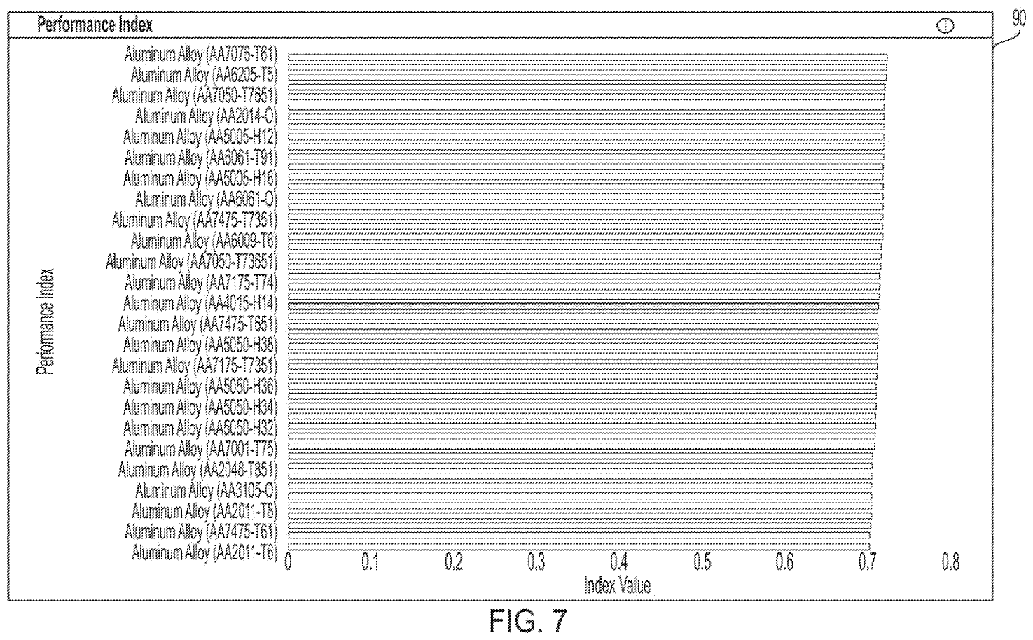

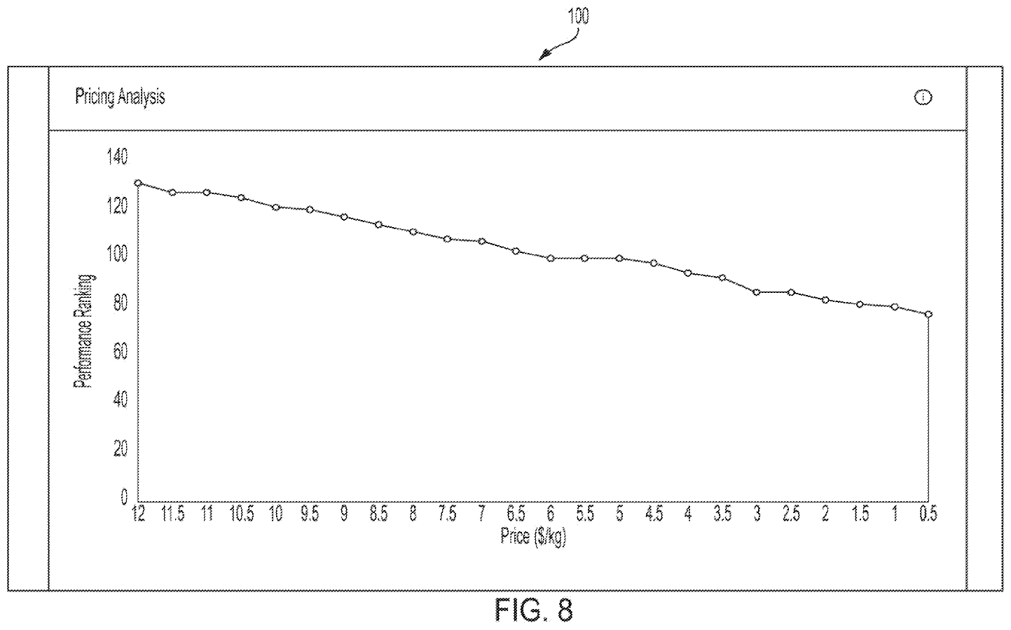

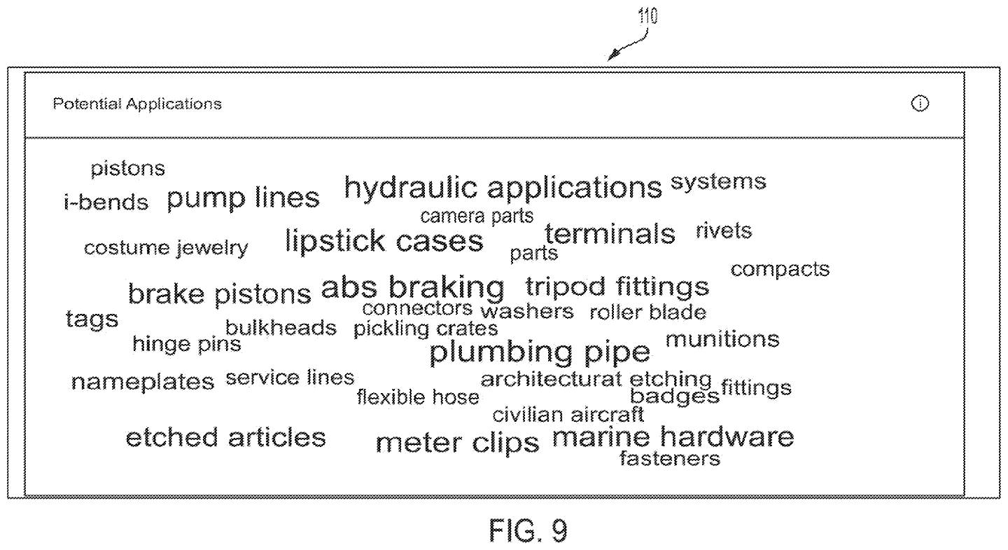

In some embodiments, a materials analysis engine 30 is provided. Referring to FIG. 5, in an analysis user interface 80 a user may enter in box 82 material properties such as price, yield strength, etc. Outputs are provided in view 84. Referring to FIG. 6, the user may assign a percentage weight to each specified property. The materials analysis engine 30 then generates a performance index for the material based on the inputs. In one example, referring to FIG. 7, the materials analysis engine compares the performance index of a proposed material to the performance indexes of existing materials in output view 90. Referring to FIG. 8, a cost comparison with similar materials may be displayed in output view 100. A degree of value of a potential new material may be ascertained in this way. In another example, based on input properties, a set of predicted commercial applications of the proposed material is generated. This may be displayed as a word cloud 110 as illustrated in FIG. 9.

For each of the use cases, materials may be ranked on properties evaluated versus the properties of other materials, cost competitiveness, strength, or other properties.

In some embodiments, the AI Materials Assistant 10 uses compositions, properties, processing steps, and microstructure of proposed materials to search patent databases. Responsive patents and published applications are returned to the user. In this way, potential patentability of a proposed material or an impediment to its use may be ascertained.

What is claimed is:

1. An AI materials assistant, comprising:
a materials database, the materials database comprising compositional, manufacturing process, and physical/mechanical properties of a plurality of materials;
an optimization engine, the optimization engine further comprising:
an Artificial Intelligence (AI) engine in operative communication with the materials database, the AI engine being trained on data in the materials database and their associated compositional, manufacturing process and physical/mechanical properties; and
a searching model in operative communication with the AI engine;
at least one user input interface in operative communication with the searching model for inputting queries regarding potential materials or desired material properties; and
at least one user output interface in operative communication with the searching model for providing materials predicted by the AI engine or material properties predicted by the AI engine to users.

2. The AI materials assistant of claim 1, wherein the AI engine comprises a Machine Learning algorithm.

3. The AI materials assistant of claim 2, wherein the Machine Learning algorithm using a multivariable regressions selected from the group consisting of: linear regression, polynomial regression, logistic regression, quantile regression, Lasso regression, ridge regression, elastic net regression, principal components regression, partial least squares regression, ordinal regression, Poisson regression, negative binomial regression, quasi Poisson regression, Cox regression, Tobit regression, support vector regression, random forest regression, decision tree regression, k-nearest neighbors (KNN) regression, and Gaussian process regression.

4. The AI materials assistant of claim 2, wherein the Machine Learning algorithm using Gaussian process multivariable regression.

5. The AI materials assistant of claim 2, wherein the AI engine comprises a Machine Learning algorithm using deep learning and/or neural network.

6. The AI materials assistant of claim 1 wherein materials are predicted by the AI engine using a random walk process on a multi-dimensional element/property map.

7. The AI materials assistant of claim 3, wherein the random walk process employs a plurality of walkers.

8. The AI materials assistant of claim 1, wherein predicted material properties include one or both groups consisting of confidence levels and error bars.

9. The AI materials assistant of claim 1, further comprising an analysis engine, the analysis engine comprising:
an analysis AI engine in operative communication with the materials database, the analysis AI engine being trained on data in the materials database and their associated compositional, manufacturing process and physical/mechanical properties; and
an analysis model in operative communication with the AI engine;
at least one user input interface in operative communication with the analysis model for inputting a plurality of material properties and a user-determined weight for each property; and
at least one user output interface in operative communication with the analysis model for providing performance indexes for a plurality materials to users based on materials properties in the materials database and the user-determined weights for the material properties.
